(19) United States
(12) Patent Application Publication
(10) Pub. No.: US 2004/0043990 A1
(43) Pub. Date: Mar. 4, 2004

(54) Method of treatment

(76) Inventor: Karen Jackson, Sheffield (GB)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/410,311

(22) Filed: Apr. 9, 2003

Related U.S. Application Data
(63) Continuation-in-part of application No. 10/349,431, filed on Jan. 22, 2003.

(30) Foreign Application Priority Data
Apr. 9, 2002 (GB) 0208129.7

(51) Int. Cl. A61K 31/7052; A61K 31/5513; A61K 31/485

(52) U.S. Cl. 514/221; 514/23; 514/282

(57) ABSTRACT

There is described a method of treatment of a patient requiring analgesic therapy which comprises the administration of an analgesically effective amount of devazepide.

There is also described the use of devazepide in the manufacture of an analgesically effective medicament.

This invention relates to a novel method of treatment and to a novel use of a medicament.

International Patent Application No. WO 99/18967 describes pharmaceutical compositions for treating chronic and neuropathic pain which comprises an analgesic amount of an opioid and an opioid potentiating amount of a CCK antagonist. WO '967 describes the use of both CCK-A (CCK-1) antagonists and CCK-B (CCK-2) antagonists, although it is described that, generally, CCK-B (CCK-2) antagonists are preferred. Moreover, page 2, lines 6 to 8 of WO '967 describes that CCK-A (CCK-1)-antagonists may be suitable, but only at relatively higher dosages.

One specific CCK-A (CCK-1) antagonist which is mentioned is devazepide, which is 3s-(−)1,3-dihydro-3-(2-indolecarbonylamino)-1-methyl-5-phenyl-2H-1,4-benzodiazepin-2-one.

Xu, in “Pain 1994; 56:271-277”, describes the effect of pharmacological intervention on a specific type of pain called allodynia, modelled in the rat. Allodynia refers to pain from stimuli which are not normally painful and pain which occurs other than in the area stimulated. It is not synonymous with referred pain. Allodynia is a (clinical) condition in which a normally painless stimulus like touch, warmth, or coolness is perceived as painful.

Xu reported a specific pain response, which was presumed to be allodynic, that responded to a CCK-B (CCK-2) antagonist in the absence of opioids. This was thought to be mediated by endogenous opioids, particularly since the response was reversed by the opioid receptor antagonist, naloxone. However, importantly, the pain response observed by Xu did not respond to a CCK-A (CCK-1) antagonist.

International Patent Application No. WO 99/18967 describes a pharmaceutical formulation comprising a CCK antagonist, such as devazepide (Devacade®), an opioid and a biphasic carrier, comprising a glyceride derivative organic phase. The use of the CCK antagonist is intended to block the CCK receptors thereby reversing or preventing the development of opioid tolerance in patients and potentiating the analgesic effect of the opioid.

However, in clinical studies we have surprisingly found that, in some patients, opioid therapy was able to be removed completely. These patients experienced pain relief on administration of devazepide only. Thus it is a novel aspect of the present invention to be able to use devazepide as an analgesic therapy in its own right, rather than solely as an adjunct to opioid therapy.

Thus, according to the invention we provide a method of treatment of a patient requiring analgesic therapy which comprises the administration of an analgesically effective amount of devazepide.

In particular, we provide a method of treatment as hereinbefore described characterised in that the patient is requiring analgesic therapy to treat or mitigate neuropathic pain.

Although the object of the present invention is to provide a method of treatment using devazepide alone, the method may nevertheless comprise the administration of devazepide in association with conventionally known analgesic therapies. Such therapies may include, for example, conventionally known opioids. However, in this aspect of the invention it should be understood that the devazepide will have a specific analgesic therapeutic effect.

According to a further feature of the invention we provide a method of treatment of a patient requiring analgesia which comprises the administration of a therapeutically effective amount of an opioid analgesic and the separate, simultaneous or sequential administration of an analgesically effective amount of devazepide.

In this aspect of the invention a variety of opioids may be used. Thus, the opioid may be selected from those which are effective analgesics and particularly those which need to be administered at relatively high or increasing doses. Examples include morphine, or a salt thereof such as the sulphate, chloride or hydrochloride, or the other 1,4-hydroxymorphinan opioid analgesics such as meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these. Naloxone is also included within the definition of an opioid. Especially preferred analgesics which may be mentioned are hydromorphone, oxycodone, morphine, e.g. morphine sulphate and fentanyl. In a preferred embodiment of the invention the analgesic is morphine or morphine sulphate. In a further preferred embodiment the opioid is fentanyl or a salt thereof.

In the method of the invention the devazepide and/or the opioid may be administered using any methods conventionally known per se. Thus, such methods would include, but shall not be limited to, administration intravenously, intra-arterially, orally, intrathecally, intranasally, intrarectally, intramuscularly/subcutaneously, by inhalation and by transdermal patch. When the devazepide and/or opioid is administered intravenously, it may, for example, be as an intravenous bolus or a continuous intravenous infusion. When the devazepide and/or the opioid is administered subcutaneously, it may for example be by subcutaneous infusion. Preferably, the opioid and/or devazepide are administered intravenously or orally. Oral administration is especially preferred. In a further preferred embodiment the opioid may be administered by a transdermal patch. When a transdermal patch is used, the preferred opioid is fentanyl or a salt thereof.

Preferentially, the opioid and the devazepide will be administered using the same mode of administration. Thus, for example, when the opioid is administered intravenously then the devazepide will be administered intravenously also. Similarly, when the opioid is administered orally then the devazepide will be administered orally also. However, it is within the scope of the invention for either the opioid to be administered orally and the devazepide to be administered intravenously or vice versa.

In this method a patient may be started on an analgesic therapy which comprises administering an effective amount of an opioid in conjunction with devazepide. As the treatment becomes effective the opioid dose may be diminished, eventually to zero, e.g. wherein devazepide is the sole therapy.

In a further embodiment, if a pain episode occurs when a patient is on a devazepide only treatment, e.g. an analgesically effective amount of devazepide, an additional dose of an opioid may, optionally, be administered.

According to a further aspect of the invention we provide the use of devazepide in the manufacture of an analgesically effective medicament.

In the use of the invention it should be understood that the devazepide will have an analgesic therapeutic effect. We especially provide the use as hereinbefore described wherein the medicament is effective in the treatment or alleviation of neuropathic pain.

In a preferred embodiment of the invention the use comprises use of devazepide as the sole active ingredient in the medicament.

Thus, in the method of the invention the daily dosage of devazepide may vary depending upon, inter alia, the weight of the patient, the method of administration, etc. In patients that are suffering serious disorders, such as cancer patients, the weight of the patient may be very low and therefore the dosage of devazepide consequentially may be low. Thus the daily dosage of devazepide may be up to 0.7 mg/kg/day. Preferably, the daily dosage of devazepide may be from 25 μg/kg/day to 0.7 mg/kg/day, more preferably from 50 μg/kg/day to 0.5 mg/kg/day. For oral administration the daily dosage of devazepide may be from 0.07 mg/kg/day to 0.7 mg/kg/day, preferably 0.07 mg/kg/day to 0.29 mg/kg/day. For intravenous administration the dosage of devazepide is preferably 50 μg/kg/day to 0.5 mg/kg/day.

In the method of the invention the dosage of the opioid analgesic administered may vary depending upon, inter alia, the nature of the opioid analgesic, the weight of the patient, the method of administration, etc. Thus, for example, the dosage of, e.g. an opioid, such as morphine, may be from 5 to 2000 mg daily. A particular dosage which may be mentioned is from 10 to 240 mg daily. A daily dosage of morphine may be from 5 to 100 mg or occasionally up to 500 mg.

When the composition used in the method of the invention includes a filler, the composition may generally comprise devazepide and a surfactant, in the ratio as hereinbefore described, with the remainder of the composition being made up with a filler.

A preferred embodiment of the invention comprises a method wherein a composition as hereinbefore described is filled into a capsule. Any conventionally known materials may be used for the capsule, however a preferred material is gelatin.

Thus, for example, in one embodiment of the invention the composition may be made up into a capsule formulation, e.g. with a fill weight of 150 mg±5% by weight or 300 mg±5% by weight. In the one preferred embodiment, the capsule formulation may comprise 1.25 mg devazepide, and 148.75 mg of a filler or other excipients, e.g. corn starch. In a further preferred embodiment, the capsule formulation may comprise 2.5 mg devazepide, and 297.5 mg of a filler or other excipients, e.g. corn starch.

Thus, such fillers may be selected from the group lactose, mannitol, talc, magnesium stearate, sodium chloride, potassium chloride, citric acid, spray-dried lactose, hydrolysed starches, directly compressible starch, microcrystalline cellulose, cellulosics, sorbitol, sucrose, sucrose-based materials, icodextrin, calcium sulphate, dibasic calcium phosphate and dextrose. A preferred filler is starch, e.g. corn starch.

When the composition of the invention includes a filler, the size of the devazepide and filler particles may be the same or different. However, in a preferred embodiment the sizes of the devazepide and filler particles will differ. Preferentially, the devazepide and/or the filler may be of reduced particle size, e.g. by milling.

The devazepide used in the method of the invention is the S enantiomer, preferentially, the S enantiomer wherein the level of R enantiomer, which may be present as an impurity, is not greater than 1.5% w/w.

The invention will now be illustrated by way of example only and with reference to the accompanying drawings in which;

FIG. 1 is a graph comparing respective dosages of devazepide and opioid (morphine) over a given period of time; and

FIG. 2 is a graph comparing respective dosages of devazepide and opioid (dihydrocodeine) over a given period.

### EXAMPLE 1

Clinical Assessment Study

A research programme has included a double blind, double dummy, randomised, crossover study of a single dose of either 1.25 mg devazepide, 5.0 mg devazepide or placebo. Patients who took part in the study had pain with a neuropathic element, and were taking regular, stable doses of strong opioids. Following completion of the study those patients who, in the opinion of their Clinical Investigator, had gained benefit from participation were given the opportunity to consent to continue receiving devazepide treatment for a period of up to six months.

Study Design

This continuation study was a multicentre, open label study of devazepide at twice daily doses of 1.25 mg, 2.5 mg and 5.0 mg.

Study Objective

The primary objective of this study was to compare descriptive and visual analogue scale (VAS) assessments of pain and pain relief in patients with neuropathic pain.

Methods

At the end of the previous randomised trial, patients received 1.25 mg devazepide twice daily for an initial period of one week. After this initial one week period, the dose of devazepide was reviewed and increased, if necessary, to 2.5 mg twice daily and thereafter to 5.0 mg twice daily as required. Devazepide treatment was continued for a period of up to six months.

During the study patients were required to remain on stable, regular doses of opioids at a dose prescribed by the investigator.

Study Assessments

Patients were assessed at clinic visits at week 1, week 2 (dose escalation) and thereafter at routine monthly clinic visits.

At weekly intervals for the first eight weeks and at monthly intervals thereafter, patients recorded pain and global pain relief using VAS and descriptive pain questionnaires. The questionnaires were returned to the Investigator at the monthly visits.

At each clinic visit the Investigator assessed safety and the patients' pain relief, reviewed the dosage, and decided if devazepide treatment should be continued.

Results

Seventeen patients elected to stay on devazepide by entering the continuation study and received devazepide at 1.25 mg, 2.5 mg or 5.0 mg twice daily for up to 26 weeks.

Of these patients, ten appeared to achieve long-term pain relief (5-26 weeks) with devazepide. Despite the requirement to remain on stable, regular doses of opioids at the dose prescribed by the investigator, several patients reduced markedly or reduced to zero their daily opioid dose.

The graph of FIG. 1 illustrates the reduction in opioid (morphine) dosage which may be achieved by administration of devazepide over a period of five months. The patient(s) commenced on 50 mg morphine per day.

FIG. 2 illustrates the trend with a weaker opioid, dihydrocodeine. The patient(s) commenced on 120 mg dihydrocodeine per day.

1. A method of treatment of a patient requiring analgesic therapy which comprises the administration of an analgesically effective amount of devazepide.

2. A method of treatment according to claim 1 characterised in that the patient is requiring analgesic therapy to treat or mitigate neuropathic pain.

3. A method according to claim 1 characterised in that the method comprises intravenous administration of devazepide.

4. A method according to claim 1 characterised in that the method comprises oral administration of devazepide.

5. A method of treatment of a patient requiring analgesic therapy which comprises the administration of a therapeutically effective amount of an opioid analgesic and the separate, simultaneous or sequential administration of an analgesically effective amount of devazepide.

6. A method according to claim 5 characterised in that the opioid is selected from the group morphine, or a salt thereof such as the sulphate, chloride or hydrochloride, or the other 1,4-hydroxymorphinan opioid analgesics such as meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these, or a combination of the aforementioned compounds.

7. A method according to claim 5 characterised in that the opioid is naloxone.

8. A method according to claim 6 characterised in that the analgesic is selected from the group hydromorphone, oxycodone, morphine and fentanyl or a salt of any of these.

9. A method according to claim 8 characterised in that the opioid is fentanyl or a salt thereof.

10. A method according to claim 8 characterised in that the opioid is selected from the group morphine and morphine sulphate.

11. A method according to claim 5 characterised in that the treatment comprises initial analgesic dosages of an opioid and devazepide.

12. A method according to claim 11 characterised in that the treatment comprises;
(i) initial analgesic dosages of an opioid and devazepide; and
(ii) subsequent dosages of devazepide only.

13. A method according to claims 5 characterised in that some pain episodes may be treated by administering an additional dosage or dosages of an opioid.

14. A method according to claim 1 characterised in that the treatment comprises;
(i) initial analgesic doses of devazepide only; and
(ii) subsequent analgesic dose(s) of an opioid and devazepide.

15. A method according to claim 1 characterised in that the method of delivery of the devazepide and/or the opioid is selected from the group, administration intravenously, intra-arterially, orally, intrathecally, intranasally, intrarectally, intramuscularly/subcutaneously, by inhalation and by transdermal patch.

16. A method according to claim 15 characterised in that the opioid and the devazepide are administered using the same mode of administration.

17. A method according to claim 15 characterised in that the devazepide and/or the opioid is administered intravenously.

18. A method according to claim 17 characterised in that the intravenous administration is by intravenous bolus or a continuous intravenous infusion.

19. A method according to claim 15 characterised in that the devazepide and/or the opioid is administered subcutaneously.

20. A method according to claim 19 characterised in that the subcutaneous administration is as a subcutaneous infusion.

21. A method according to claim 15 characterised in that the devazepide and/or the opioid is administered orally.

22. A method according to claim 12 characterised in that the devazepide is administered orally.

23. A method according to claim 17 characterised in that the opioid is administered intravenously and the devazepide is administered intravenously.

24. A method according to claim 21 characterised in that the opioid is administered orally and the devazepide is administered orally.

25. A method according to claim 15 characterised in that the opioid is administered by intravenous administration or oral administration.

26. A method according to claim 25 characterised in that the composition comprises devazepide and a surfactant with the remainder of the composition being made up with a filler.

27. A method according to claims 1 characterised in that the composition is filled into a capsule.

28. A method according to claim 27 characterised in that the capsule is a gelatin capsule.

29. A method according to claim 15 characterised in that the opioid is administered by transdermal patch.

30. A method according to claim 29 characterised in that the opioid is fentanyl, or a salt thereof.

31. A method according to claim 1 characterised in that the daily dosage of devazepide is up to 0.7 mg/kg/day.

32. A method according to claim 31 characterised in that the daily dosage of devazepide is from 25 μg/kg/day to 0.7 mg/kg/day.

33. A method according to claim 32 characterised in that the daily dosage of devazepide is from 50 μg/kg/day to 0.5 mg/kg/day.

34. A method according to claim 31 characterised in that the dosage of devazepide is an oral dosage.

35. A method according to claim 34 characterised in that for oral administration the daily dosage of devazepide is from 0.07 mg/kg/day to 0.7 mg/kg/day.

36. A method according to claim 34 characterised in that the devazepide is administered orally and the daily dosage of devazepide is from 0.07 mg/kg/day to 0.29 mg/kg/day.

37. A method according to claim 17 characterised in that the devazepide is administered intravenously and the daily dosage of devazepide is from 50 μg/kg/day to 0.5 mg/kg/day.

38. A method according to claim 5 characterised in that the daily dosage of the opioid is from 5 to 2000 mg daily.

39. A method according to claim 38 characterised in that the dosage of the opioid is from 10 to 240 mg daily.

40. A method according to claim 38 characterised in that the daily dosage of the opioid is from 5 to 100 mg daily.

41. A method according to claim 1 characterised in that the devazepide used in the method of the invention is substantially the S enantiomer.

42. A method according to claim 41 characterised in that the level of R enantiomer, which may be present as an impurity, is not greater than 1.5% w/w.

43. The use of devazepide in the manufacture of an analgesically effective medicament.

44. The use according to claim 31 characterised in that the medicament is effective in the treatment or alleviation of neuropathic pain.

45. The use according to claim 43 characterised in that the method comprises intravenous administration of devazepide.

46. The use according to claim 43 characterised in that the method comprises oral administration of devazepide.

47. The use of treatment of a patient requiring analgesic therapy which comprises the administration of a therapeutically effective amount of an opioid analgesic and the separate, simultaneous or sequential administration of an analgesically effective amount of devazepide.

48. The use according to claim 47 characterised in that the opioid is selected from the group morphine, or a salt thereof such as the sulphate, chloride or hydrochloride, or the other 1,4-hydroxymorphinan opioid analgesics such as meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these, or a combination of the aforementioned compounds.

49. The use according to claim 48 characterised in that the opioid is naloxone.

50. The use according to claim 48 characterised in that the analgesic is selected from the group hydromorphone, oxycodone, morphine and fentanyl, or a salt of any of these.

51. The use according to claim 50 characterised in that the opioid is fentanyl or a salt thereof.

52. The use according to claim 50 characterised in that the opioid is selected from the group morphine and morphine sulphate.

53. The use according to claim 47 characterised in that the treatment comprises initial analgesic dosages of an opioid and devazepide.

54. The use according to claim 53 characterised in that the treatment comprises;
(i) initial analgesic dosages of an opioid and devazepide; and
(ii) subsequent dosages of devazepide only.

55. The use according to claims 47 characterised in that some pain episodes may be treated by administering an additional dosage or dosages of an opioid.

56. The use according to claim 431 characterised in that the treatment comprises;
(i) initial analgesic doses of devazepide only; and
(ii) subsequent analgesic dose(s) of an opioid and devazepide.

57. The use according to claim 43 characterised in that the method of delivery of the devazepide and/or the opioid is selected from the group, administration intravenously, intra-arterially, orally, intrathecally, intranasally, intrarectally, intramuscularly/subcutaneously, by inhalation and by transdermal patch.

58. The use according to claim 57 characterised in that the opioid and the devazepide are administered using the same mode of administration.

59. The use according to claim 57 characterised in that the devazepide and/or the opioid is administered intravenously.

60. The use according to claim 59 characterised in that the intravenous administration is by intravenous bolus or a continuous intravenous infusion.

61. The use according to claim 57 characterised in that the devazepide and/or the opioid is administered subcutaneously.

62. The use according to claim 61 characterised in that the subcutaneous administration is as a subcutaneous infusion.

63. The use according to claim 57 characterised in that the devazepide and/or the opioid is administered orally.

64. The use according to claim 54 characterised in that the devazepide is administered orally.

65. The use according to claim 59 characterised in that the opioid is administered intravenously and the devazepide is administered intravenously.

66. The use according to claim 63 characterised in that the opioid is administered orally and the devazepide is administered orally.

67. The use according to claim 57 characterised in that the opioid is administered by intravenous administration or oral administration.

68. The use according to claim 67 characterised in that the composition comprises devazepide and a surfactant with the remainder of the composition being made up with a filler.

69. The use according to claims 43 characterised in that the composition is filled into a capsule.

70. The use according to claim 69 characterised in that the capsule is a gelatin capsule.

71. The use according to claim 57 characterised in that the opioid is administered by transdermal patch.

72. The use according to claim 71 characterised in that the opioid is fentanyl, or a salt thereof.

73. The use according to claim 43 characterised in that the daily dosage of devazepide is up to 0.7 mg/kg/day.

74. The use according to claim 73 characterised in that the daily dosage of devazepide is from 25 μg/kg/day to 0.7 mg/kg/day.

75. The use according to claim 74 characterised in that the daily dosage of devazepide is from 50 μg/kg/day to 0.5 mg/kg/day.

76. The use according to claim 73 characterised in that the dosage of devazepide is an oral dosage.

77. The use according to claim 76 characterised in that for oral administration the daily dosage of devazepide is from 0.07 mg/kg/day to 0.7 mg/kg/day.

78. The use according to claim 76 characterised in that the devazepide is administered orally and the daily dosage of devazepide is from 0.07 mg/kg/day to 0.29 mg/kg/day.

79. The use according to claim 59 characterised in that the devazepide is administered intravenously and the daily dosage of devazepide is from 50 μg/kg/day to 0.5 mg/kg/day.

80. The use according to claim 47 characterised in that the daily dosage of the opioid is from 5 to 2000 mg daily.

81. The use according to claim 80 characterised in that the dosage of the opioid is from 10 to 240 mg daily.

82. The use according to claim 80 characterised in that the daily dosage of the opioid is from 5 to 100 mg daily.

83. The use according to claim 43 characterised in that the devazepide used in the method of the invention is substantially the S enantiomer.

84. The use according to claim 83 characterised in that the level of R enantiomer, which may be present as an impurity, is not greater than 1.5% w/w.
